(19) United States
(12) Patent Application Publication
(10) Pub. No.: US 2026/0229357 A1
(43) Pub. Date: Aug. 6, 2026

(54) DEEP LEARNING-BASED LANGUAGE PROCESSING DEVICE, DEEP LEARNING-BASED LANGUAGE PROCESSING METHOD, AND NON-TRANSITORY COMPUTER-READABLE MEDIUM

(71) Applicant: NEC Corporation, Tokyo (JP)

(72) Inventor: Yutaka UNO, Tokyo (JP)

(73) Assignee: NEC Corporation, Tokyo (JP)

(21) Appl. No.: 19/150,929

(22) PCT Filed: Feb. 1, 2023
(86) PCT No.: PCT/JP2023/003189

§ 371 Date: Jul. 25, 2025

(51) Int. Cl. G16H 50/20; G16H 10/60

(52) U.S. Cl. CPC G16H 50/20; G16H 10/60

(57) ABSTRACT

In order to accurately generate a meaning expression by more sufficiently taking in context across a plurality of documents, this language processing device comprises: an acquisition unit which acquires, in a document column, a partial string including a first document; a context information generation unit which generates context information pertaining to a first element with reference to the first element composing the first document and a second element composing each second document other than the first document included in the partial string; and a meaning information generation unit which generates meaning information corresponding to the first element with reference to the context information. The deep learning-based language processing device can support decision-making of users such as medical professionals.

### TECHNICAL FIELD

The present invention relates to natural language processing.

### BACKGROUND ART

Deep learning-based language processing models are known. For example, BERT disclosed in NPL 1 is a language processing model that generates meaning expression based on context for each token included in an input sentence. BERT is constituted by Transfomer's encoder and has a Self-Attention mechanism that refers to sentences and adjacent sentences to incorporate context in the meaning expression.

### Citation List

### Non Patent Literature

NPL 1: Jacob Devlin et al., “BERT: Pre-training of Deep Bidirectional Transformers for Language Understanding”, NAACL 2019.

### SUMMARY OF INVENTION

### Technical Problem

Here, the context of the sentence may exist not only in the document including the sentence but also in other documents. For example, in a plurality of documents related to each other, one document may include incomplete sentences, expressions with different interpretations, and the like. Such incomplete sentences and expressions with different interpretations may be complemented or interpreted uniquely by referring to other related documents. In such cases, there is context across multiple documents. The BERT disclosed in NPL 1 can incorporate context based on input sentences and adjacent sentences, but cannot incorporate context across a plurality of documents. Therefore, there is a problem that when there is a context across a plurality of documents, meaning expressions cannot be accurately generated from each document.

One aspect of the present invention has been made in view of the above problems, and an object of the present invention is to provide a technique for generating meaning expression with high accuracy by sufficiently incorporating context across a plurality of documents.

### Solution to Problem

A language processing device according to an aspect of the present invention includes: an acquisition means for acquiring a partial string including a first document in a document column; a context information generation means for referring to a first element constituting the first document and a second element constituting each of second documents other than the first document included in the partial string to generate context information regarding the first element; and a meaning information generation means for generating meaning information corresponding to the first element with reference to the context information.

A language processing method according to an aspect of the present invention includes: acquiring, by at least one processor, a partial string including a first document in a document column; generating context information regarding the first element with reference to a first element constituting the first document and a second element constituting each of second documents other than the first document included in the partial string; and generating meaning information corresponding to the first element with reference to the context information.

A program according to an aspect of the present invention causes a computer to function as: an acquisition means for acquiring a partial string including a first document in a document column; a context information generation means for referring to a first element constituting the first document and a second element constituting each of second documents other than the first document included in the partial string to generate context information regarding the first element; and a meaning information generation means for generating meaning information corresponding to the first element with reference to the context information.

### Advantageous Effects of Invention

According to one aspect of the present invention, it is possible to generate the meaning expression with high accuracy by sufficiently incorporating context across a plurality of documents.

### BRIEF DESCRIPTION OF DRAWINGS

FIG. 1 is a block diagram illustrating a configuration of a language processing device according to a first example embodiment.

FIG. 2 is a flowchart illustrating a flow of a language processing method according to the first example embodiment.

FIG. 3 is a schematic diagram illustrating a specific example of a document column in a second example embodiment.

FIG. 4 is a block diagram illustrating a configuration of a language processing device according to the second example embodiment.

FIG. 5 is a schematic diagram illustrating an attention mechanism included in a transformer for comparison with the second example embodiment.

FIG. 6 is a schematic diagram illustrating Self-Attention for comparison with the second example embodiment.

FIG. 7 is a schematic diagram illustrating an improvement of a context information generation unit in the second example embodiment.

FIG. 8 is a flowchart illustrating a flow of a language processing method according to the second example embodiment.

FIG. 9 is a schematic diagram illustrating a further improvement of a context information generation unit in a modified example of the second example embodiment.

FIG. 10 is a block diagram illustrating a hardware configuration example of a language processing device constituting each example embodiment.

### EXAMPLE EMBODIMENT

### First Example Embodiment

A first example embodiment of the present invention will be described in detail with reference to the drawings. The present example embodiment is a basic form of the example embodiment described below.

### Configuration of Language Processing Device 1

A configuration of a language processing device 1 according to the present example embodiment will be described with reference to FIG. 1. FIG. 1 is a block diagram illustrating a configuration of the language processing device 1. As illustrated in FIG. 1, the language processing device 1 includes an acquisition unit 11, a context information generation unit 12, and a meaning information generation unit 13. The acquisition unit 11 acquires a partial string including a first document in a document column. The context information generation unit 12 refers to a first element constituting the first document and a second element constituting each of second documents other than the first document included in the partial string to generate context information regarding the first element. The meaning information generation unit 13 refers to the context information and generates meaning information corresponding to the first element.

Here, the document includes a set of sentences described in a natural language, and is a unit of data stored in a storage device independently of other documents. For example, the document may be a file or a record of a database, but is not limited thereto.

### Implementation Example by Program

In a case where the language processing device 1 is configured by a computer, a program according to the present example embodiment is stored in a memory of the computer. The program causes the computer to function as: an acquisition unit 11 configured to acquire a partial string including a first document in a document column; a context information generation unit 12 configured to refer to a first element constituting the first document and a second element constituting each of second documents other than the first document included in the partial string to generate context information regarding the first element; and a meaning information generation unit 13 configured to generate meaning information corresponding to the first element with reference to the context information.

### Flow of Language Processing Method S1

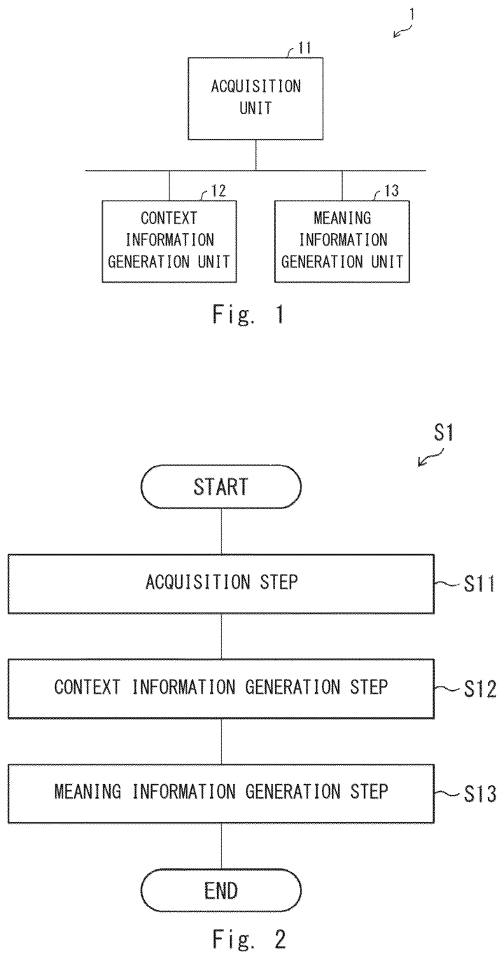

The language processing device 1 configured as described above executes the language processing method S1 according to the present example embodiment. A flow of a language processing method S1 will be described with reference to FIG. 2. FIG. 2 is a flowchart illustrating the flow of the language processing method S1. As illustrated in FIG. 2, the language processing method S1 includes an acquisition step S11, a context information generation step S12, and a meaning information generation step S13. In the acquisition step S11, the acquisition unit 11 acquires a partial string including the first document in the document column. In the context information generation step S12, the context information generation unit 12 refers to the first element constituting the first document and the second element constituting each of second documents other than the first document included in the partial string to generate context information regarding the first element. In the meaning information generation step S13, the meaning information generation unit 13 generates meaning information corresponding to the first element with reference to the context information.

As described above, in the present example embodiment, a configuration is adopted in which, the partial string including the first document is acquired in the document column, the context information regarding the first element is generated with reference to the first element constituting the first document and the second element constituting each of the second documents other than the first document included in the partial string, and the meaning information corresponding to the first element is generated with reference to the context information. Therefore, according to the present example embodiment, it is possible to obtain an effect that the meaning expression can be generated with high accuracy by sufficiently capturing the context across a plurality of documents.

### Second Example Embodiment

A second example embodiment of the present invention will be described in detail with reference to the drawings. Components having the same functions as the components described in the first example embodiment are denoted by the same reference signs, and the description thereof will be appropriately omitted.

### Document Column

The document columns in the present example embodiment are documents recorded with respect to a predetermined thing, and constitute a time series based on the order in which each document is recorded. Hereinafter, “medical care” is applied as a predetermined thing, and “medical examination article” which is an example of “medical record recorded in medical care” is applied as a document.

### Problems in the Present Example Embodiment

The medical examination article is also referred to as an electronic medical chart, and is an electronic medical record recorded by one or more medical workers such as doctors and nurses. Here, since the medical examination article is used mainly for the purpose of improving the efficiency of medical treatment fee request, inspection, or prescription order, the description content itself of the medical examination article is not sufficiently utilized. As a result, there are some examples in which the medical examination article is merely memos by the recording person himself/herself. The medical examination article recorded as memos often includes incomplete sentences, expressions with different interpretations, and the like. On the other hand, from the viewpoint of applying a deep learning-based language processing model to medical care, it is conceivable to create a preliminary learning model in the medical field by utilizing medical examination articles. However, as described above, it is not possible to generate accurate meaning information from incomplete sentences and expressions with different interpretations only by referring to medical examination articles recorded as memos alone. In the present example embodiment described below, meaning information in the medical field can be generated with high accuracy by utilizing medical examination articles recorded as memos.

### Medical Record Database

Each medical examination article is stored in a medical record database (not illustrated) in association with the recording time. Furthermore, each medical examination article may be stored in association with an item related to the examination, such as a recorder, a patient, or the like, in addition to the recording time.

For example, the medical record database may be constituted by a relational database. In this case, as an example, the medical record database may include a medical examination article table, a recorder table, and a patient table. For example, the medical examination article table includes a record including a medical examination article using a combination of recording time, identification information of a recorder, and identification information of a patient as a key. In addition, the recorder table includes the attribute of the recorder using the identification information of the recorder as a key. In addition, the patient table includes the attribute of the patient using the identification information of the patient as a key.

The above-described time series of medical examination articles is acquired as a result of searching such a medical record database using a predetermined condition. For example, as a result of searching using the condition “specific patient”, a time series of medical examination articles recorded for the specific patient is acquired. In addition, as a result of the search using the condition of “specific recorder”, a time series of medical examination articles recorded by the specific recorder is acquired. Furthermore, for example, as a result of the search using the condition “patient having a specific attribute”, a time series of medical examination articles recorded for a patient having a specific attribute is acquired. For example, as a result of the search using the condition “recorder having a specific attribute”, the time series of medical examination articles recorded by the recorder having the specific attribute is acquired. Note that specific examples of the time series of the medical examination articles are not limited to these examples.

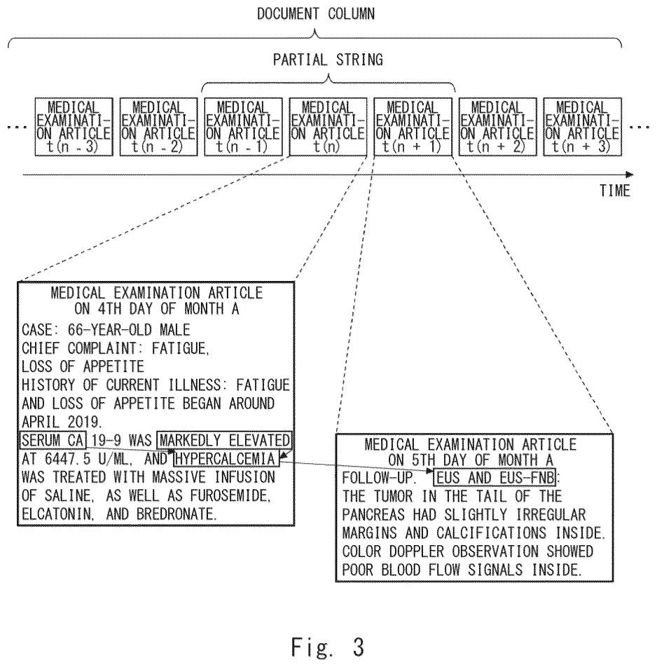

The specific examples of the time series of the medical examination articles will be described with reference to FIG. 3. FIG. 3 is a schematic diagram illustrating a specific example of the time series of the medical examination articles (an example of a document column). As illustrated in FIG. 3, the time series of the medical examination articles includes medical examination articles t(i) (i=1, 2, . . . , n, . . . ). i indicates an order based on the recording time, and a larger value indicates a newer recording.

As an example, the medical examination article t(n) is recorded for a patient of “66-year-old male” on “4th day of month A”. In addition, the “hypercalcemia” included in the medical examination article t(n) is a description related to “serum CA” and “marked elevation”. In addition, the medical examination article t(n+1) was recorded for the patient on “5th day of month A”. The “EUS, EUS-FNB” included in the medical examination article t(n+1) is a description related to “hypercalcemia” included in the previous medical examination article t(n). Thus, the context of the description exists across a plurality of medical examination articles t (i).

In addition, in the example of FIG. 3, the medical examination articles t(n−1), t(n), and t(n+1) constitute a partial string in time series of the medical examination articles. The partial string is referred to for generating context information. Hereinafter, the partial strings are also simply referred to as “medical examination article strings to be referred to”. The medical examination article t(n) is a target for generating the meaning information of the token and is an example of a first document described in the claims. The medical examination articles t(n−1) and t(n+1) are medical examination articles other than the medical examination article t(n) in the medical examination article string to be referred to, and are an example of a second document described in the claims.

### Configuration of Language Processing Device 1A

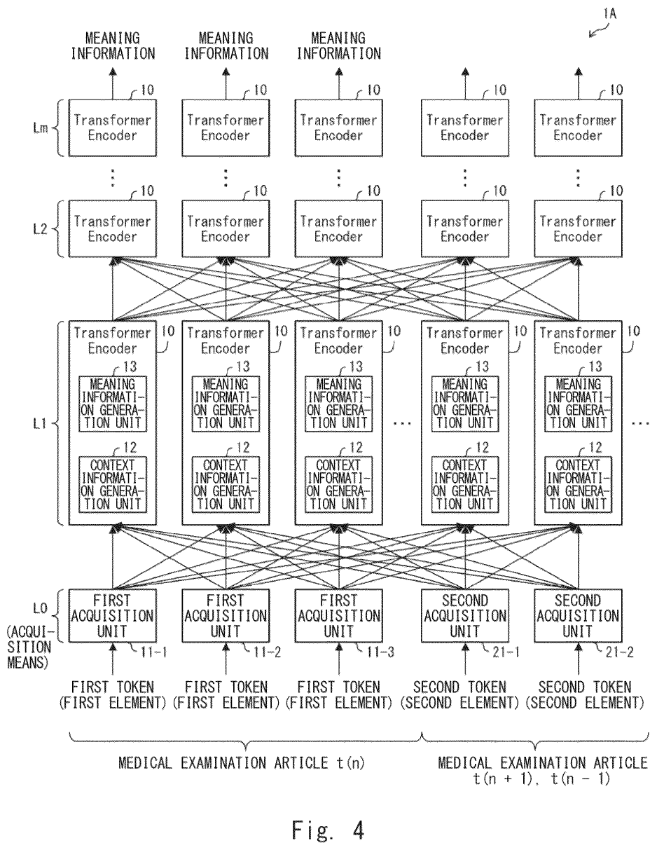

A configuration of a language processing device 1A according to the present example embodiment will be described with reference to FIG. 4. FIG. 4 is a block diagram illustrating the configuration of the language processing device 1A. As illustrated in FIG. 4, the language processing device 1A includes layers L0, L1, L2, . . . , and Lm (m is a natural number). When information based on the medical examination articles t(n), t(n+1), and t(n−1) is input to the layer L0, meaning information of each token included in the medical examination article t(n) is output from the layer Lm via the layers L1, L2, . . . .

The meaning information is information indicating a meaning feature of each token, and is also referred to as meaning expression, distributed expression, embedded vector, or the like. The meaning information output from the layer Lm corresponds to each token, and is information in which context across the medical examination articles t(n), t(n+1), and t(n−1) is incorporated.

The language processing device 1A further includes first acquisition units 11-1, 11-2, 11-3, . . . (hereinafter, each of them is also referred to as a first acquisition unit 11A) and second acquisition units 21-1, 21-2, . . . (hereinafter, each of them is also referred to as a second acquisition unit 21A). The first acquisition unit 11A and the second acquisition unit 21A are arranged in the layer L0. The layer L0 is an example of a configuration that implements the acquisition means described in the claims. In addition, the language processing device 1A includes a stack of encoders 10 connected to the first acquisition units 11A and the second acquisition units 21A. The encoder 10 includes a context information generation unit 12 and a meaning information generation unit 13, and has a configuration obtained by improving an Encoder of a known Transformer. Each of the stacked encoders 10 is arranged in any one of the layers L1, L2, . . . , and Lm. The encoder 10 arranged in the layer Lk(k=1, 2, . . . , m−1) processes information input from the layer L_k−1 and outputs the processed information to the layer L_k+1.

### First Acquisition Unit 11A

The first acquisition unit 11A acquires input information corresponding to a token constituting the medical examination article t(n). For example, the input information includes input embedding, location embedding, and segment embedding. The input embedding is an initial value of the meaning information corresponding to the token. The position embedding indicates a position of the token in the medical examination article t(n). The segment embedding indicates the medical examination article t(n) including the token. Note that the token is also called a morpheme, for example, and is a minimum unit constituting the document. The token constituting the medical examination article t(n) is an example of a first element described in the claims. Hereinafter, the token as an example of the first element is also referred to as a first token. By causing each of the first acquisition units 11A included in the layer L0 to function, the input information corresponding to the string of the first token in the medical examination article t(n) is acquired.

### Second Acquisition Unit 21A

The second acquisition unit 21A acquires input information corresponding to tokens constituting the medical examination articles t(n+1) and t(n−1). Since the input information is described in the same manner as the input information acquired by the first acquisition unit 11A, detailed description cannot be obtained. The token constituting each of the medical examination articles t(n+1) and t(n−1) is an example of a second element described in the claims. Hereinafter, the token as an example of the second element is also referred to as a second token. By causing each of the second acquisition units 21A included in the layer L0 to function, the input information corresponding to the string of the second token in the medical examination articles t(n+1) and t(n−1) is acquired.

### Context Information Generation Unit 12

The context information generation unit 12 refers to a plurality of first tokens and a plurality of second tokens to generate context information corresponding to each of the first tokens. For example, the context information corresponding to a certain first token is information including the attention weight of each of the other first tokens with respect to the first token and the attention weight of each of the second tokens. More specifically, the context information corresponding to a certain first token is information obtained by integrating the input information corresponding to the first token and each attention weight described above. For example, the context information generation unit 12 is achieved by improving an attention mechanism in the Encoder of the Transformer. Details of the context information generation unit 12 will be described later.

### Meaning Information Generation Unit 13

The meaning information generation unit 13 generates meaning information corresponding to each token with reference to the context information generated by the context information generation unit 12. The generated meaning information is obtained by adding context information. For example, the meaning information generation unit 13 is achieved by a feedforward network (FFN) connected to a rear stage of an attention mechanism in an Encoder of a Transformer.

### Details of Context Information Generation Unit 12

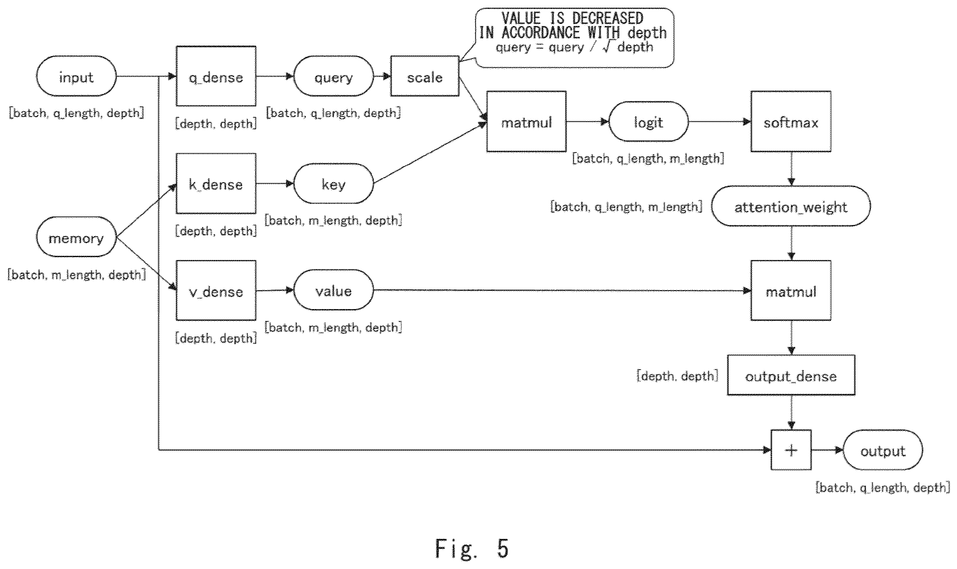

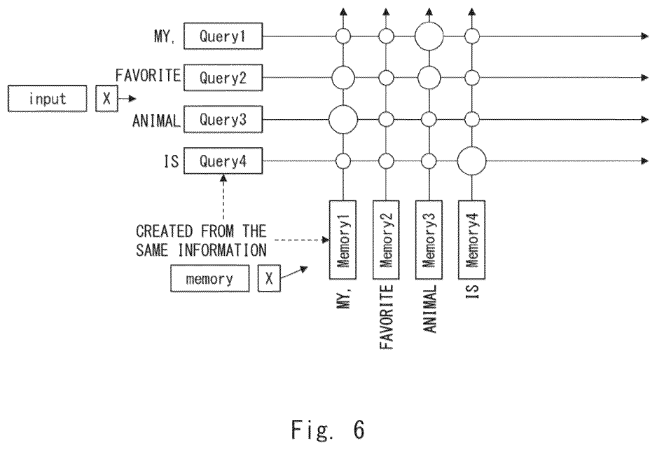

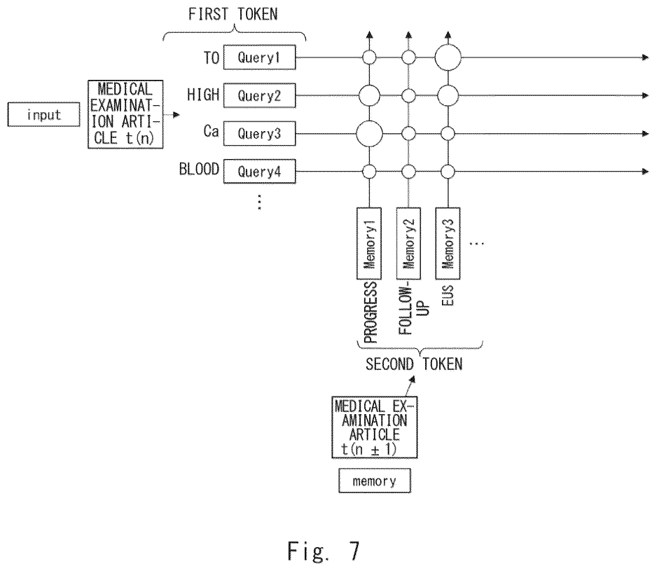

Details of the context information generation unit 12 will be described. As described above, the context information generation unit 12 can be achieved by improving the attention mechanism of the encoder of the transformer. Hereinafter, first, the attention mechanism of the Transformer will be described with reference to FIGS. 5 and 6, and next, improvements of the context information generation unit 12 on the attention mechanism will be described with reference to FIG. 7.

FIG. 5 is a schematic diagram illustrating an attention mechanism included in the encoder of the Transformer for comparison with the context information generation unit 12 in the present example embodiment. As illustrated in FIG. 5, the attention mechanism generates an output with reference to an input and a memory. Here, the input indicates a token string. In addition, the memory indicates a string of tokens for capturing a context related to the input. The attention mechanism sets input information corresponding to each token of the input as a query, and sets input information corresponding to each token of the memory as a key and a value. Furthermore, the attention mechanism calculates the attention weight of each value for each query by obtaining a matrix product (matmal) of the query and the key. Furthermore, the attention mechanism generates an output (output) by obtaining a matrix product (matmal) of the attention weight and the value. The attention mechanism of the encoder of the Transformer is Self-Attention that refers to a string of the same token as the input as a memory. Self-Attention will be described with reference to FIG. 6 in order to compare with the context information generation unit 12 of the present example embodiment described later. Note that Self-Attention is also adopted in BERT (NPL 1) using only the Encoder of the Transformer.

FIG. 6 is a schematic diagram illustrating Self-Attention for comparison with the context information generation unit 12 in the present example embodiment. As illustrated in FIG. 6, in Self-Attention, a string (in this example, “my, favorite, animal, is”) of the same token created from the same information X is referred to as an input and a memory.

Here, in FIG. 6, a line segment with a rightward arrow having each query (Queries 1 to 4) as an end point schematically illustrates processing performed for each query. Furthermore, a line segment with an upward arrow with each memory (memories 1 to 4) as an end point schematically indicates processing performed for each memory (memories 1 to 4). In addition, a circle on an intersection of these line segments indicates the attention weight, and the larger the area, the higher the attention weight. Therefore, as can be seen from FIG. 6, in a case where the length of the string of the tokens is 4, processing of obtaining the attention weight is generated for 4×4=16 combinations of the input and the memory. That is, in Self-Attention, the calculation cost for capturing the context in one sentence increases in proportion to the square of the length of the sentence.

On the other hand, FIG. 7 is a schematic diagram illustrating an improvement of the context information generation unit 12 with respect to the attention mechanism described above. As illustrated in FIG. 7, the context information generation unit 12 refers to the input information corresponding to the string of the first token “to, high, Ca, blood, . . . ” (queries 1, 2, 3, 4, . . . ) included in the medical examination article t(n) as the input. Furthermore, the context information generation unit 12 refers to input information corresponding to a string of the second token “progress, follow-up, EUS, . . . ” (memories 1, 2, 3, . . . ) included in the medical examination articles t(n+1) and t(n−1) as a memory. It is assumed that the memories 1, 2, 3, . . . include a key and a value, respectively. Note that, in FIG. 7, the line segments having the respective queries and the respective memories as end points and the circles on the intersections of the line segments are as described with reference to FIG. 6, and thus the detailed description thereof will not be repeated. As can be seen from FIG. 7, in a case where the length of the string of the first token is 4 and the length of the string of the second token is 3, in the context information generation unit 12, processing of obtaining the attention weight is generated for 4×3=12 combinations of the input and the memory.

Here, a case where the context of the medical examination article t(n) and the medical examination articles t(n+1) and t(n−1) is to be incorporated using the Self-Attention illustrated in FIG. 6 will be considered. In this case, in Self-Attention, the input information corresponding to the string of the token obtained by concatenating the string of the first token and the string of the second token is referred to as an input and a memory. In this example, the length of the string of coupled tokens is 7, and processing of obtaining the attention weight is generated for 7×7=49 combinations of the input and the memory. That is, the calculation cost of the processing for incorporating the context across the plurality of medical examination articles increases according to the square (for example, 49) of the sum of the lengths of the medical examination articles in Self-Attention. On the other hand, in the context information generation unit 12 according to the present example embodiment, the calculation cost can be suppressed to an extent of increasing according to the product (for example, 12) of the lengths of the medical examination articles. Therefore, the context information generation unit 12 can greatly reduce the calculation cost for generating the meaning information in which the context across the plurality of medical examination articles is incorporated, as compared with the technique disclosed in NPL 1 using Self-Attention.

### Flow of Language Processing Method S1A

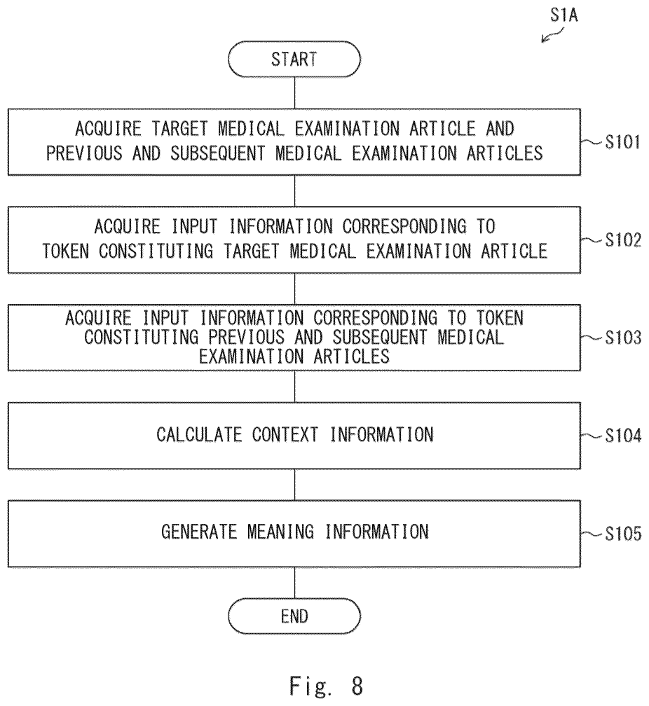

The language processing device 1A configured as described above executes the language processing method S1A according to the present example embodiment. The flow of the language processing method S1A will be described with reference to FIG. 8. FIG. 8 is a flowchart illustrating the flow of the language processing method S1A. As illustrated in FIG. 8, the language processing method S1A includes steps S101 to S105.

In step S101, the language processing device 1A acquires the target medical examination article t(n) and the medical examination articles t(n+1) and t(n−1) previous and subsequent the target medical examination article t(n) as medical examination article strings to be referred to from the medical record database.

In step S102, the first acquisition unit 11A acquires the input information corresponding to the first token constituting the target medical examination article t(n).

In step S103, the second acquisition unit 21A acquires the input information corresponding to the second token constituting the previous and subsequent medical examination articles t(n+1) and t(n−1).

In step S104, the context information generation unit 12 generates context information corresponding to each first token by referring to input information corresponding to the first token as a query and referring to input information corresponding to the second token as a memory.

In step S105, the meaning information generation unit 13 generates meaning information corresponding to each first token with reference to the context information. Thereafter, the meaning information generated in step S105 in the layer Lk−1 is input to the layer Lk, and steps S104 to S105 are repeatedly executed. Then, the meaning information of each first token is output from the layer Lm.

### Effects of Present Example Embodiment

In the present example embodiment, in addition to the configuration similar to the first example embodiment, a configuration is adopted in which the documents included in the document column are medical examination articles, each medical examination article forms a time series based on the order in which the medical examination articles are recorded, and each medical examination article is stored in the medical record database in association with the recording time. Therefore, according to the present example embodiment, in addition to the effects obtained by the first example embodiment, in a case where a new medical examination article is often recorded as memos based on the medical examination article recorded previously, it is possible to incorporate the context of a plurality of medical examination articles into the meaning expression of each token, and it is possible to generate meaning information with high accuracy.

### First Modified Example

In the second example embodiment described above, an example has been described in which the second element constituting the medical examination articles t(n+1) and t(n−1) (an example of the second document) is the same unit (token) as the first element. The present invention is not limited thereto, and the second element may be an element constituting the second document in a unit larger than the first element. For example, the second element may be a sentence, a paragraph, or a document larger than the token. In this case, the present modified example is similarly described by replacing the “second token” in the description of the second example embodiment with a sentence, a paragraph, or a document. As a result, the number of second elements to be referred to as a memory by the context information generation unit 12 can be reduced, and the number of combinations of a query and a memory as illustrated in FIG. 7 can be further reduced. Furthermore, the context can be incorporated in a larger unit as compared with a case where the same token as the first element is adopted as the second element. Therefore, in the modified example, it is possible to generate the meaning information with high accuracy by capturing the context of the previous and subsequent medical examination articles while suppressing the calculation cost.

### Second Modified Example

In the second example embodiment described above, an example has been described in which three medical examination article strings to be referred to for generating the context information are the medical examination article of the target and the medical examination articles previous and subsequent the medical examination article. However, the medical examination article string to be referred to for generating the context information is not limited thereto. For example, the medical examination article strings to be referred to may be two of the medical examination article of the target and the previous medical examination article (or the subsequent medical examination article). Note that “before” in the time series of medical examination articles refers to an older one, and “after” refers to a newer one. Even in this case, the context of the medical examination article recorded at least in the before or the after can be incorporated, so that the meaning information of the token can be accurately generated.

### Third Modified Example

In addition, the medical examination article string to be referred to for generating the context information may include a target medical examination article t(n) to a document separated by two or more previous and subsequent the medical examination article t(n) in the time series of the medical examination article. For example, the medical examination article string to be referred to may be one or both of documents separated from the medical examination article t(n) up to N1 documents before and documents separated from the medical examination article t(n) up to N2 documents after the medical examination article t(n). N1 and N2 are natural numbers, and may be the same value or different values. As a specific example in a case where N1 and N2 are the same value, the medical examination article strings to be referred to may be five articles of a medical examination article t(n), medical examination articles t(n+1) and t(n−1) previous and subsequent the medical examination article t(n), and medical examination articles t(n+2) and (n−2) separated by two previous and subsequent the medical examination article t(n). In addition, the medical examination article string to be referred to may include one or both of a medical examination article recorded within a predetermined period before the recording time of the medical examination article t(n) and a medical examination article recorded within a predetermined period after the recording time, and the medical examination article t(n). Examples of the predetermined period include one day, one week, one month, and the like, but are not limited thereto.

In the present modified example, in addition to the components illustrated in FIG. 4, the language processing device 1A further includes a second acquisition unit 21A corresponding to each medical examination article separated by two or more from the medical examination article t(n), and a stack of encoders 10 connected to the second acquisition unit 21A. Details of the added second acquisition unit 21 A and encoder 10 are as described above.

The present modified example corresponds to a case where the context of medical examination articles extends not only to the previous and subsequent medical examination articles but also to two or more medical examination articles separated from each other, and it is possible to generate the meaning information of the token with high accuracy by incorporating in the context from a plurality of medical examination articles in a wider range.

### Fourth Modified Example

Furthermore, the above-described third modified example may be further modified, and the second element may be an element constituting the second document in a larger unit as the second document including the second element is farther from the first document in the medical examination article string to be referred to.

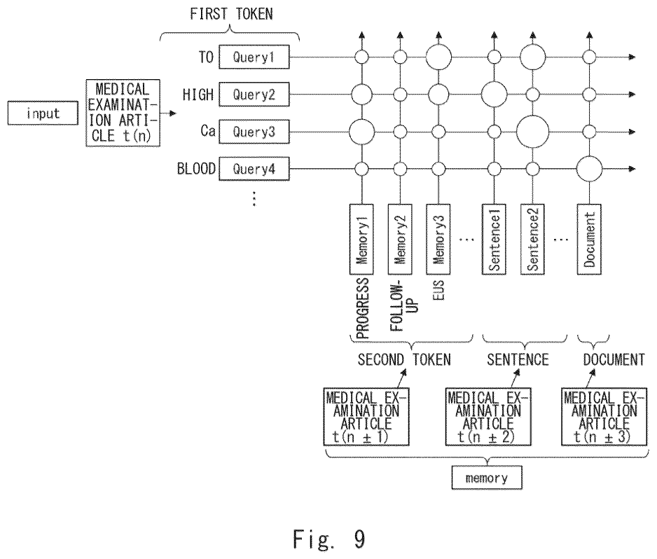

A specific example of the present modified example will be described with reference to FIG. 9. FIG. 9 is a schematic diagram illustrating a further improvement of the context information generation unit 12 according to the present modified example. In the example of FIG. 9, the medical examination article string to be referred to for generating the context information includes seven medical examination articles up to the medical examination articles t(n+3) and t(n−3) separated by three previous and subsequent the target medical examination article t(n).

In this case, in addition to the components illustrated in FIG. 4, the language processing device 1A further includes a second acquisition unit 21A corresponding to the medical examination articles t(n+2) and t(n−2), a second acquisition unit 21A corresponding to the medical examination articles t(n+3) and t(n−3), and a stack of encoders 10 connected to these second acquisition units 21A. The second acquisition unit 21A corresponding to the medical examination articles t(n+2) and t(n−2) acquires input information corresponding to sentences constituting the medical examination articles t(n+2) and t(n−2). The second acquisition unit 21A corresponding to the medical examination articles t(n+3) and t(n−3) acquires input information corresponding to the medical examination articles t(n+3) and t(n−2) themselves (documents).

As illustrated in FIG. 9, the context information generation unit 12 refers to the input information corresponding to the string of the first token included in the medical examination article t(n) as an input similarly to the example of FIG. 7. Further, the context information generation unit 12 refers to the input information corresponding to the string of the second token included in the medical examination articles t(n+1) and t(n−1) as a memory similarly to the example of FIG. 7. Further, the context information generation unit 12 refers to input information corresponding to a sentence (Sentence 1, Sentence 2, . . . ) included in the medical examination articles t(n+2) and t(n−2) as a memory. Further, the context information generation unit 12 refers to input information corresponding to the medical examination articles t(n+3) and t(n−3) themselves (Document) as a memory.

In the example of FIG. 9, each of the second token, the sentence, and the document is an example of a second element described in the claims, and is a larger unit as the medical examination article including the second element is farther from the medical examination article t(n). Note that the input information corresponding to the sentence may be obtained by integrating meaning information (alternatively, meaning information including context) corresponding to each second token included in the sentence. Furthermore, the input information corresponding to the document may be obtained by integrating meaning information (alternatively, meaning information including context) corresponding to each second token included in the document. Such “meaning information including context” may be meaning information separately generated by the language processing device 1A according to the present modified example using the medical examination articles t(n+2), (n−2), (n+3), and (n−3) themselves, which are the second documents, as the target medical examination articles. Furthermore, the input information corresponding to the sentence or the document may be meaning information generated based on a known technique using the sentence or the document as a unit.

Here, it is considered that the relevance between the medical examination article as the second document and the medical examination article t(n) as the first document decreases as the distance increases. Therefore, in the present modified example, by incorporating in the context in a coarser unit as the distance increases, it is possible to generate the meaning information of the token with high accuracy while suppressing an increase in calculation cost while referring to a plurality of medical examination articles in a wider range.

### Other Modified Examples

Furthermore, in the second example embodiment described above, an example in which each document stored in the medical record database is a medical examination article has been described. However, other types of medical records may be stored in the medical record database. Specific examples of other types of medical records include, but are not limited to, inspection records, image interpretation records, nursing records, and the like. Furthermore, the plurality of documents referred to by the context information generation unit 12 may include different types of medical records.

In addition, in the above-described example embodiment, a document in a field other than the medical record may be applied as each document included in the document column. In this case, each document may be a document recorded with respect to a predetermined thing, and the document column may constitute a time series based on the order in which each document is recorded. Examples of the predetermined thing include, but are not limited to, records related to education, records related to growth of animals and plants, records related to production of goods, and the like. In this case, it is conceivable that the context related to a predetermined thing extends across a plurality of documents. In such a case, the present modified example can incorporate the context across the plurality of documents into the meaning expression of each token, and can generate the meaning information with high accuracy.

### Example of Implementation by Software

Some or all of the functions of the language processing devices 1 and 1A (hereinafter, also referred to as “each of the above devices”) may be achieved by hardware such as an integrated circuit (IC chip) or may be achieved by software.

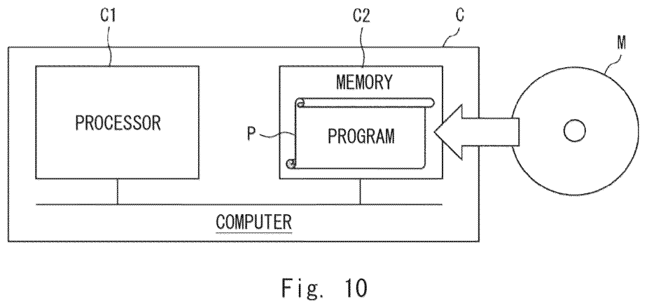

In the latter case, the above device is implemented by, for example, a computer that executes a command of a program which is software for implementing each function. An example of such a computer (hereinafter, referred to as a computer C) is shown in FIG. 10. The computer C includes at least one processor C1 and at least one memory C2. A program P for causing the computer C to operate as the above device is recorded in the memory C2. In the computer C, the processor C1 reads the program P from the memory C2 and executes the program P to implement each function of the above apparatuses.

As the processor C1, for example, a central processing unit (CPU), a graphic processing unit (GPU), a digital signal processor (DSP), a micro processing unit (MPU), a floating point number processing unit (FPU), a physics processing unit (PPU), a tensor processing unit (TPU), a quantum processor, a microcontroller, or a combination thereof can be used. As the memory C2, for example, a flash memory, a hard disk drive (HDD), a solid state drive (SSD), or a combination thereof can be used.

Note that the computer C may further include a random access memory (RAM) for developing the program P at the time of execution and temporarily storing various types of data. In addition, the computer C may further include a communication interface for transmitting and receiving data to and from other apparatuses. The computer C may further include an input/output interface for connecting input/output devices such as a keyboard, a mouse, a display, and a printer.

In addition, the program P can be recorded in a non-transitory tangible recording medium M readable by the computer C. As such a recording medium M, for example, a tape, a disk, a card, a semiconductor memory, a programmable logic circuit, or the like can be used. The computer C can acquire the program P via such a recording medium M. In addition, the program P can be transmitted via a transmission medium. As such a transmission medium, for example, a communication network, a broadcast wave, or the like can be used. The computer C can also acquire the program P via such a transmission medium.

### Supplementary Information 1

The present disclosure is not limited to the above-described example embodiments, and various alterations can be made within the scope described in the claims. For example, example embodiments obtained by appropriately combining the technical means disclosed in the above-described example embodiments are also included in the technical scope of the present invention.

### Supplementary Information 2

Some or all the above-described example embodiments may be described as the follows. However, the present invention is not limited to the following aspects.

### Supplementary Note 1

A language processing device including:
an acquisition means for acquiring a partial string including a first document in a document column;
a context information generation means for referring to a first element constituting the first document and a second element constituting each of second documents other than the first document included in the partial string to generate context information regarding the first element; and
a meaning information generation means for generating meaning information corresponding to the first element with reference to the context information.

### Supplementary Note 2

The language processing device according to Supplementary Note 1, wherein
each document included in the document column is a document recorded regarding a predetermined object, and
the document column constitutes a time series based on an order in which each document is recorded.

### Supplementary Note 3

The language processing device according to Supplementary Note 2, wherein
each document included in the document column is a medical record recorded with respect to medical care, and
each medical record is stored in the medical record database in association with a record time.

### Supplementary Note 4

The language processing device according to any one of Supplementary Notes 1 to 3, wherein the second element is an element constituting the second document in a unit larger than the first element.

### Supplementary Note 5

The language processing device according to any one of Supplementary Notes 1 to 4, wherein the partial string includes from the first document to a document two or more documents before or after the first document in the document column.

### Supplementary Note 6

The language processing device according to Supplementary Note 5, wherein the second element is an element constituting the second document in a larger unit as the second document including the second element is farther from the first document in the partial string.

### Supplementary Note 7

A language processing method including, by at least one processor:
acquiring a partial string including a first document in a document column;
referring to a first element constituting the first document and a second element constituting each of second documents other than the first document included in the partial string to generate context information regarding the first element; and
generating meaning information corresponding to the first element with reference to the context information.

### Supplementary Note 8

A program for causing a computer to function as:
an acquisition means for acquiring a partial string including a first document in a document column;
a context information generation means for referring to a first element constituting the first document and a second element constituting each of second documents other than the first document included in the partial string to generate context information regarding the first element; and
a meaning information generation means for generating meaning information corresponding to the first element with reference to the context information.

### Supplementary Note 9

A language processing device including:
at least one processor,
wherein the processor is configured to perform
an acquisition process of acquiring a partial string including a first document in a document column;
a context information generation process of referring to a first element constituting the first document and a second element constituting each of second documents other than the first document included in the partial string to generate context information regarding the first element; and
a meaning information generation process of generating meaning information corresponding to the first element with reference to the context information.

Note that the language processing device may further include a memory, and the memory may store a program for causing the processor to execute the acquisition process, the context information generation process, and the meaning information generation process. In addition, this program may be recorded in a computer-readable non-transitory tangible recording medium.

### REFERENCE SIGNS LIST

1, 1A language processing device
10 encoder
11 acquisition unit
11-1, 11-2, 11-3 first acquisition unit
21-1, 21-2 second acquisition unit
12 context information generation unit
13 meaning information generation unit
C1 processor
C2 memory

What is claimed is:

1. A deep learning-based language processing device comprising:
at least one memory configured to store instructions; and
at least one processor configured to execute the instructions to:
acquire medical examination article strings including at least a medical examination article t(n) at a predetermined timing and a medical examination article t(i) at a different timing;
refer, in an attention mechanism in an Encoder of a Transformer, to a string of first tokens constituting the medical examination article t(n) as input and a string of second tokens constituting the medical examination article t(i) as memory to generate context information regarding the string of first tokens, wherein the context information is information including the attention level of the string of second tokens relative to the string of first tokens; and
generate meaning information corresponding to the string of first tokens with reference to the context information.

2. The deep learning-based language processing device according to claim 1, wherein
the medical examination article strings constitute a time series based on an order in which the medical examination article t(n) and the medical examination article t(i) are recorded.

3. The deep learning-based language processing device according to claim 2, wherein
the medical examination article t(n) and the medical examination article t(i) included in the medical examination article strings are stored in the medical record database in association with a record time.

4. The deep learning-based language processing device according to claim 1, wherein the string of second tokens is a string of tokens constituting the medical examination article t(i) in a unit larger than the string of first tokens.

5. The deep learning-based language processing device according to claim 1, wherein the medical examination article strings includes from the medical examination article t(n) to a document two or more documents before or after the medical examination article t(n) in the medical examination article strings.

6. The deep learning-based language processing device according to claim 5, wherein the string of second tokens is an string of second tokens constituting the medical examination article t(i) in a larger unit as the medical examination article t(i) including the string of second tokens is farther from the medical examination article t(n) in the medical examination article strings.

7. A deep learning-based language processing method comprising, by at least one processor:
acquiring medical examination article strings including at least a medical examination article t(n) at a predetermined timing and a medical examination article t(i) at a different timing;
referring, in an attention mechanism in an Encoder of a Transformer, to a string of first tokens constituting the medical examination article t(n) as input and a string of second tokens constituting the medical examination article t(i) as memory to generate context information regarding the string of first tokens, wherein the context information is information including the attention level of the string of second tokens relative to the string of first tokens; and
generating meaning information corresponding to the string of first tokens with reference to the context information.

8. A non-transitory computer-readable medium stored with a program for causing a computer to execute processes of:
acquiring medical examination article strings including at least a medical examination article t(n) at a predetermined timing and a medical examination article t(i) at a different timing;
referring, in an attention mechanism in an Encoder of a Transformer, to a string of first tokens constituting the medical examination article t(n) as input and a string of second tokens constituting the medical examination article t(i) as memory to generate context information regarding the string of first tokens, wherein the context information is information including the attention level of the string of second tokens relative to the string of first tokens; and
generating meaning information corresponding to the string of first tokens with reference to the context information.
